(19) United States
(12) Patent Application Publication
(10) Pub. No.: US 2013/0059906 A1
(43) Pub. Date: Mar. 7, 2013

(54) METHODS AND COMPOSITIONS FOR INFLUENCING TUMORS USING MICRORNA-185 AS A TUMOR SUPPRESSOR

(71) Applicants: Manjeet K. Rao, San Antonio, TX (US); J. Saadi Imam, El Paso, TX (US)

(72) Inventors: Manjeet K. Rao, San Antonio, TX (US); J. Saadi Imam, El Paso, TX (US)

(73) Assignee: The Board of Regents of the University of Texas System, Austin, TX (US)

(21) Appl. No.: 13/579,789

(22) PCT Filed: Feb. 17, 2011
(86) PCT No.: PCT/US2011/025318

§ 371 Date: Oct. 28, 2012

Related U.S. Application Data
(60) Provisional application No. 61/305,417, filed on Feb. 17, 2010.

(51) Int. Cl. A61K 31/7105; A61P 35/04; C12N 15/113; A61P 35/00; C12N 5/071; C12N 5/09

(52) U.S. Cl. 514/44 R; 435/375; 536/24.5

(57) ABSTRACT

The present invention is directed to the use of microRNA-185 (miR-185) as a tumor suppressor. Embodiments of the invention are directed to the use of miR-185 to modulate the expression of tumor-causing genes and to treat subjects.

### CROSS-REFERENCES TO RELATED APPLICATIONS

This application claims priority from U.S. Provisional Patent Application No. 61/305,417, filed Feb. 17, 2010, which is hereby incorporated by reference as if fully set forth herein.

### TECHNICAL FIELD OF THE INVENTION

The invention is directed to the use of microRNA-185 (miR-185) as a tumor suppressor.

### BACKGROUND OF THE INVENTION

Genes are biomolecular units in cells that carry information for the function of the cell. Cells are the basic units making up organisms, such as human beings. Genes for a cell are located on chromosomes in the nucleus of the cell. Genes are made of deoxyribonucleic acid (“DNA”). DNA is a made up of two complementary strands of polymers made up of four nucleic acids, adenine (“A”), guanine (“G”), cytosine (“C”), and thymine (“T”). A gene contains introns and exons. The exons encode a protein. When the gene is expressed, the gene is transcribed into messenger RNA (“mRNA”), followed by translation of the mRNA into protein. RNA is made up of one strand of a polymer made up of A, G, C, and uracil (“U”). An mRNA is translated into the protein encoded by the gene from which the mRNA has been transcribed. Homeobox genes are genes that encode proteins that can bind to sequences DNA. Thus, homeobox genes play a role in controlling transcription.

Tumor growth and metastasis is partly derived from deregulation of genes that are critical regulators of normal developmental and differentiation. One group of genes that plays a crucial role in both development and tumorigenesis is the homeobox gene family. One homeobox gene of interest is the sine oculis related homeobox 1 homolog (Six1) homeoprotein. Sixq1 is deregulated in multiple human tumors and is associated with poor patient survival. Six1 is a potent tumor causing gene (oncogene) that is known to play central role in the development of poor outcome, aggressive, metastatic adult human cancers including breast cancer, ovarian cancer, hepatocellular carcinoma, as well as pediatric malignancies such as rhabdomyosarcoma and Wilms' tumor.

Much is known about the function of homeobox genes. For example, Six1 plays an important role during development and frequent deregulation in a large number of neoplasms. Six1 plays an important role in the expansion of progenitor cell populations during early embryogenesis and is known to be essential for the development of numerous organs. In addition, Six1 is reported to be overexpressed in multiple pediatric and adult human cancers, including breast, ovarian, cervical, hepatocellular carcinomas as well as rhabdomyosarcomas and Wilms' tumors. Importantly, Six1 overexpression has been strongly associated with aggressive, metastatic cancers and poor prognosis. Recently, Six1 overexpression in immortalized mammary epithelial cells has been shown to induce transformation, leading to highly aggressive and invasive tumors in nude mice.

Relatively little is known about the regulation of homeobox genes, particularly in cancers. For example, in spite of Six1's important role during development and frequent deregulation in a large number of neoplasms, little is known about its regulation.

MicroRNAs are naturally-occurring short non-coding sequences of RNA. MicroRNA's were discovered about 15 years ago and are still being investigated. One role of microRNA is to bind to mRNA. mRNA has a coding region which is followed by a 3′ untranslated region (“3′ UTR”). A complementary mRNA sequence binds an mRNA sequence in the 3′ UTR. Thus, one role for mRNA is controlling translation.

MicroRNAs (miRNAs) are believed to be involved in many biological processes including normal development and differentiation. Most individual microRNA are thought to target multiple genes. Recent developments have suggested an important role for miRNAs in cancer growth and metastasis. miRNAs have recently been shown to regulate expression of several genes involved in tumorigenesis and increasing evidence suggests that they can function as tumor suppressors or oncogenes.

### SUMMARY OF THE INVENTION

Embodiments of the invention are directed to methods of modulating gene expression in a cell, comprising administering to the cell isolated and/or chemically synthesized miR-185 in an amount sufficient to modulate the expression of a tumor-causing gene.

An embodiment of the invention is directed to methods of treating a patient in need thereof, comprising administering to the patient in need thereof, a pharmaceutical formulation comprising isolated and/or chemically synthesized miR-185 in a therapeutically effective amount sufficient to modulate cellular expression of a tumor-causing gene.

A further embodiment of the invention is directed to methods of treating a patient in need thereof, comprising administering to the patient in need thereof, a pharmaceutical formulation comprising isolated and/or chemically synthesized miR-185 in a therapeutically effective amount sufficient to modulate cellular expression of a tumor-causing gene.

### BRIEF DESCRIPTION OF THE DRAWINGS

The following description will be better understood when read in conjunction with the appended drawings. It should be understood, however, that the present invention is not limited to the precise arrangements and instrumentalities shown herein. The components in the drawings are not necessarily to scale, emphasis instead being placed upon clearly illustrating the principles of the present invention. Moreover, in the drawings, like reference numerals designate corresponding parts throughout the several views.

The invention may take physical form in certain parts and arrangement of parts. For a more complete understanding of the present invention, and the advantages thereof, reference is now made to the following descriptions taken in conjunction with the accompanying drawings, in which:

FIGS. 1A and 1B illustrate that miR-185 expression correlates reciprocally with Six1 gene expression in multiple human cancer lines and tumor tissues;

FIGS. 1C-1F illustrate that miR-185 targets the 3′UTR of the Six1 homeobox gene;

FIGS. 2A and 2B illustrate the effect of miR-185 on cell proliferation.

FIGS. 3A-3D illustrate that miR-185 inhibits colony formation in vitro and tumor growth in vivo; and

FIGS. 4A-4D illustrates that miR-185 alters cell-cycle progression and sensitizes cells to apoptosis.

### DETAILED DESCRIPTION OF EXEMPLARY EMBODIMENTS

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise.

The terms used throughout this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term.

Embodiments of the claimed invention demonstrate that microRNA-185 (miR-185) targets the Six1 homeobox gene. Further, the claimed invention identifies that miR-185 acts as a potent tumor suppressor in multiple human cancers. According to certain embodiments, the miR-185 acts as a tumor suppressor when present in an amount sufficient to modulate the expression of a tumor-causing gene. According to some embodiments, the amount is at least 10 nM miR-185 per 2,000,000 tumor cells.

According to some embodiments, the miR-185 is administered to a cell. According to an embodiment of the invention, the miR-185 is administered to a subject in need of treatment. In this embodiment, a pharmaceutical formulation comprising miR-185 is administered to a test subject in need thereof, in a therapeutically effective amount sufficient to modulate cellular expression of a tumor-causing gene. In certain embodiments of the claimed invention, a pharmaceutical formulation comprising miR-185 is administered to a test subject in need thereof, in a therapeutically effective amount sufficient to ameliorate the symptoms of a disease.

As used herein the terms “administration,” “administering,” or the like, when used in the context of providing a pharmaceutical or nutraceutical composition to a subject generally refers to providing to the subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered by parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, pharmaceutical compositions, formulations and preparations may include pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

As used herein the term “pharmaceutically acceptable salts” includes salts prepared from by reacting pharmaceutically acceptable non-toxic bases or acids, including inorganic or organic bases, with inorganic or organic acids. Pharmaceutically acceptable salts may include salts derived from inorganic bases include aluminum, ammonium, calcium, copper, ferric, ferrous, lithium, magnesium, manganic salts, manganous, potassium, sodium, zinc, etc. Examples include the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, etc.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

As used herein the term “subject” generally refers to a mammal, and in particular to a human.

As used herein, the term “treat” generally refers to an action taken by a caregiver that involves substantially inhibiting, slowing or reversing the progression of a disease, disorder or condition, substantially ameliorating clinical symptoms of a disease disorder or condition, or substantially preventing the appearance of clinical symptoms of a disease, disorder or condition.

Terms such as “in need of treatment,” “in need thereof,” “benefit from such treatment,” and the like, when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

By “therapeutically effective amount” is meant an amount of a drug or pharmaceutical composition that will elicit at least one desired biological or physiological response of a cell, a tissue, a system, animal or human that is being sought by a researcher, veterinarian, physician or other caregiver, and/or ameliorate the symptoms of a disease.

Any suitable route of administration may be employed for providing a subject with an effective dosage of miR-185 described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, compositions may be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the compositions with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Soft gelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or soft gelatin capsules, for example, for identification or in order to characterize combinations of active compound doses, or to disguise the capsule contents for usage in clinical or other studies.

In an embodiment of the invention, a pharmaceutical formulation may comprise nanoparticles comprising miR-185. In this embodiment, the nanoparticles serve as the delivery vehicle for introducing to miR-185 to the desired region in a test subject.

For the prevention or treatment of disease, the appropriate dosage of the composition will depend on the type of disease to be treated, as defined above, the severity and course of the disease, whether the compositions are administered for preventive or therapeutic purposes, previous therapy, the patient's clinical history and response to the composition, and the discretion of the attending physician. The composition is suitably administered to the patient at one time or over a series of treatments.

According to another embodiment of the invention, the effectiveness of the composition in preventing or treating disease may be improved by administering the composition serially or in combination with another agent that is effective for those purposes, such as another anti-cancer agent. Such other agents may be present in the composition being administered or may be administered separately. The composition may be suitably administered serially or in combination with radiological treatments, whether involving irradiation or administration of radioactive substances.

The miR-185 may be chemically synthesized or purchased from commercially available sources. miR-185 is typically commercially available as part of a larger sequence of RNA, chemically synthesized to match sequences found in nature, such as those sequences available in the miRBase database. Analyses of pediatric renal tumors, aggressive ovarian cancer tissues and multiple cancer cell lines of various origins showed decreased miR-185 expression, paralleling an increase in Six1 levels. Further investigation revealed that miR-185 impedes anchorage-independent growth and cell invasion in addition to suppressing tumor growth in nude mice, implicating it to be a potent tumor suppressor. Experimental results presented herein indicate that miR-185 mediates its anti-proliferative and tumor suppressor effect by regulating the cell cycle proteins and Six1 transcriptional targets c-myc and cyclin A1. Furthermore, the present inventors found that miRNA-185 could increase the sensitivity of cancer cells to tumor necrosis factor-related apoptosis inducing ligand (TRAIL)-mediated apoptosis. This is a significant finding as Six1 overexpressing TRAIL-resistant cancers have poor outcome and TRAIL has been proposed to affect several aspects of tumorigenesis including inhibition of tumor growth and metastasis, surveillance against tumor growth and response to chemotherapy. Taken together, the experimental findings herein provide a novel insight into the function of miR-185 in regulating Six1 expression in tumor growth and metastasis and implicate miR-185 as of use in therapeutic interventions. The findings implicate miR-185 to be effective for therapeutic regimen as introduction of miRNA-185 will not only result in slower tumor growth and decreased tumor invasiveness but will also lead to increased sensitivity of cancer cells to TRAIL-induced apoptosis. The present inventors believe that the results presented herein are the first reports that microRNA-185 targets the oncogene Six1.

The potent tumor suppressor function of microRNA-185 may be exploited in treating aggressive human cancers. Since it targets Six1 oncogene, microRNA-185 may be used as an efficacious drug for treating specifically Six1 overexpressing cancers. Because Six1 is deregulated in many cancers, its ability to provide resistance to TRAIL-mediated apoptosis, a natural immune surveillance pathway against cancer that spares normal cells, may be exploited for killing of Six1 overexpressing cancer cells over normal cells. Therefore, microRNA-185 will not inhibit tumor growth and tumor invasiveness but will also cause increased sensitivity of cancer cells to TRAIL-induced apoptosis. Furthermore, lower expression of microRNA-185 in aggressive tumors may be used as a prognostic marker and may also be used to identify those patients who might benefit most from TRAIL treatment.

Thus, microRNA-185 has application as a tumor suppressor. As miR-185 is shown herein to re-sensitize cells to TRAIL-mediated apoptosis, miR-185 has application as a potent and specific pro-apoptotic cancer therapeutic agent. Additionally, as miR-185 is shown to act in part by preventing cell-cycle progression through the regulation of c-myc and cyclin-A1, it has application as a cell-cycle regulator that specifically acts through the regulation of transcriptional targets downstream of Six1 and c-myc. Thus, microRNA-185 has advantageous, specific, and potent utility.

The following working examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### WORKING EXAMPLES

The results described in the Examples identify a novel use as a tumor suppressor for miRNA, miR-185. The Examples illustrate that microRNA-185 suppresses tumor growth and metastasis by repressing Six1 oncogene in human cancers.

The results presented in the Examples identify miR-185 to be responsible for regulating Six1 expression in cancers. Though the Six1 homeobox gene is known to play a central role in the development of multiple aggressive solid tumors and is associated with poor patient survival, the mechanism by which Six1 is dysregulated in cancers has not been not known. Homeobox genes encode transcription factors that are essential for normal development and often dysregulated in cancers. The Examples describe investigations of the mechanism by which the Six1 homeobox protein, which plays a crucial role during development, is frequently deregulated in several poor outcome, aggressive, metastatic adult human cancers including breast cancer, ovarian cancer, hepatocellular carcinoma, as well as pediatric malignancies such as rhabdomyosarcoma and Wilms' tumor. The present inventors predicted that microRNA plays an important role in modulating Six1 expression in cancers. Preliminary data was acquired in the form of a microRNA microarray analysis of normal and Wilms' tumor kidney tissues, which identified microRNA-185 as one of the differentially expressed microRNA in Stage 1V tumor kidney. Cloning for the validation of Six1 as a target of microRNA-185 was carried out. The tumor suppressor activity of microRNA-185 was thoroughly tested in multiple human cancer cell lines by multiple methods and in a xenograft mouse model. The results presented in the Examples reveal that miRNA-185 translationally represses Six1 by binding to its 3′UTR. The results show that miR-185, which is located at 22q11.21, directly targets the 3′UTR of Six1 transcripts and elicits a robust translational repression. Analyses of pediatric renal tumor tissues and multiple cancer cell lines of various origins showed decreased miR-185 expression, paralleling an increase in Six1 levels. The results reveal a reciprocal relationship between Six1 and miR-185 in pediatric renal tumors and cancer cell lines from diverse lineages including breast cancer, ovarian cancer and rhabdomyosarcoma, where Six1 overexpression parallels miR-185 underexpression. Further investigation reveals that miR-185 is a potent tumor suppressor as it inhibits cell proliferation, anchorage independent growth, cell invasion as well as tumor growth in immuno-compromised mice. Further investigation reveals that miR-185 impedes anchorage-independent growth and cell migration, in addition to suppressing tumor growth in nude mice, implicating it to be a potent tumor suppressor. In addition, the results show that miR-185 mediates its anti-proliferative and tumor suppressor functions in part by suppressing expression of Six1 transcriptional targets c-myc and Cyclin A1 and by sensitizing cancer cells to apoptosis in general and TRAIL-mediated apoptosis in particular. The results indicate that miR-185 mediates its tumor suppressor function by regulating cell cycle proteins and Six1 transcriptional targets c-myc and cyclin A1. Furthermore, the results show that miR-185 sensitizes Six1 overexpressing tumor necrosis factor-related apoptosis inducing ligand (TRAIL)-resistant cancer cells to apoptosis. Taken together, the findings provide a novel insight into the mechanism of Six1 regulation in cancers and implicate miR-185 in future therapeutic interventions. The findings further support the notion that miRNAs are critical regulators of tumorigenesis. The results indicate that miR-185 regulates Six1's pro-proliferative role during tumorigenesis.

The results presented in the Examples results suggest that reduced levels of miR-185 resulting in altered expression of Six1 may in part lead to the re-initiation of developmental cell proliferative pathways in Six1 overexpressing tumors. Since Six1 is known to induce normal renal cell proliferation and mutation in the Six1 gene has been shown to be prevalent in children with renal hypodysplasia, the present inventors believe that uncontrolled Six1 expression due to significantly lower levels of its regulator miRNA/s (such as miR-185) during early kidney development may lead to persistent proliferation and neoplastic transformation of renal precursor cells resulting in tumor growth.

In agreement with this, Six1 transcriptional target cyclin A1, which is normally expressed in embryonic but not in differentiated mammary gland, when reactivated due to uncontrolled Six1 expression, results in increased proliferation of breast cancer cells. Moreover, the results presented in the Examples reveal that mir-185 alters cell proliferation by regulating the expression of cyclin A1. The results presented in the Examples suggest that reduced levels of miR-185 resulting in altered expression of Six1 may in part lead to the re-initiation of developmental cell proliferative pathways in Six1 overexpressing tumors.

The sequence of miR-185 is: uggagagaaaggcaguuccuga. It typically occurs in nature within the longer sequence: AGGGGGCGAGGGAUUGGAGAGAAAGGCAGUUCCUGAUGGUCCCCUCCCCAGGGGCUGGC UUUCCUCUGGUCCUUCCCUCCCA (e.g. hsa-mir-185 MI0000482, http://www.mirbase.org/cgi-bin/query.pl?terms=mir-185), as is known to one of ordinary skill in the art.

The Examples are based in part on the following manuscript: Imam J S, Buddavarapu K, Lee-Chang J S, Campos-Cardoso C, Camosy C, Hornsby P, and Rao M K., Oncogene; 29(35): 4971-9 (2010).

The following procedures each relate to one or more of the examples.

Cell culture. Human breast cancer cell lines (MCF7 and MDA-MB-435), ovarian cancer cell lines (SKOV3), a rhabdomyosarcoma cell line (RD) and HEK-293 cells, were cultured according to the American Type Culture Collection (ATCC) protocol. OVCAR-5 and A2780 (ovarian cancer) cells were a generous gift from Dr. Alex Bishop (UT Health Science Center at San Antonio, GCCRI).

Wilms' Tumor and Ovarian Cancer tissue samples. 80 Wilms' tumors and normal matched kidney were acquired from the Children's Oncology Group (COG), Arcadia, Calif. The samples we obtained were from a heterogeneous population of tumors that did or did not have LOH at 1p or 16q. 15 Ovarian Cancer and normal ovarian tissues were acquired from MD Anderson Cancer Center Tissue Bank.

MicroRNA microarray analysis. MiRNA microarray analysis was performed on total RNA from eight Stage 1 (low risk), Stage 4 (high risk) favorable histology tumor samples and their normal matches by LC Sciences LLC (Houston) using Sanger database version 10.0 sequences. (This microarray data is not shown in this manuscript, but resulted in our initial discovery

Plasmid construction. To generate mir-185 expression construct, ˜450 bp pri-miR-185 genomic sequence was amplified from normal kidney genomic DNA (extracted with the Red Ex N Amp kit (Sigma)) and cloned in BamHI and HindIII sites of the pSilencer 4.1 puro vector. For the pMIR-Six1 3′ UTR construct, the wild type 3′-UTR segment of the Six1 gene was amplified from genomic DNA and subcloned downstream of the Luciferase gene in the pmiR-Report vector (Ambion) at SacI and SpeI sites. To generate the miR-185 triplicate construct (3X-miR-185), an oligonucleotide containing the triplicated miR-185 binding sequence in the Six1 3′UTR (˜60 nt) was annealed and cloned in p-MIR reporter vector as described above. The mutated 2-8 nt seed sequence construct was generated by performing site-directed mutagenesis on the Six1 3′UTR construct. All constructs were validated by sequencing at the UTHSCSA sequencing core.

Transfection. For the generation of stable cells, the pSilencer-miR-185 or -scramble construct was transfected into HEK-293, MCF7 and SKOV3 cells for 48 hours, followed by puromycin selection for 14 days. Of note, miR-185 stable cell lines (HEK-293, MCF7 and SKOV3) were difficult to generate and maintain; we observed a rapid loss of ectopic miR-185 expression with each consecutive passage, likely due to reduced survival of cells highly overexpressing miR-185. For transient transfections, cell were either transfected with synthetic pre-miR-185 mimic (Ambion), miRNA control (Ambion), or miR-185 inhibitor (Qiagen). Samples were harvested 48 and 72 hours after transfection for RNA and protein, respectively.

RNA extraction and real-time quantitative PCR. Total RNA was extracted from Wilms' tumor samples, normal kidney tissue and Ovarian tumor and normal tissue and cell lines using the miRNEasy Mini kit (Qiagen). Two micrograms of total RNA was reverse transcribed using the miScript RT kit (Qiagen). Forward primers specific to each miRNA or primer sets specific to each gene of interest were individually designed and tested. Real-time PCR was performed using the miScript SYBR Green PCR kit (Qiagen) for miRNA and gene expression, according to manufacturer's protocol. Replicate reactions were run for each cDNA sample. The relative expression of each gene was quantified by measuring ΔΔCt values and normalizing against RNU19 or 18S for miRNA or gene expression, respectively.

Luciferase assays. HEK-293 cells were plated at 55% confluency in six-well plates. PmiR-Reporter constructs were co-transfected with PRL-CMV using Lipofectamine 2000 as per manufacturer's instructions (Invitrogen). Luciferase activity was measured 48 hours after transfection using the dual luciferase reporter assay system (Promega). Firefly luciferase activity was normalized to Renilla luciferase activity for each transfected well. For overexpression studies, miRNA control or pre-miR-185 mimic were transfected sequentially after transfection of luciferase constructs.

Western blot analysis. HEK-293, MCF7 and SKOV3 cells transfected with synthetic pre-miR-185 mimic, miR-185 inhibitor or miRNA control sequences were lysed and protein was quantitated. 20 and 40-μg of each sample were resolved on a 12% (w/v) SDS-PAGE gel and transferred to a Millipore nylon membrane. Blots were probed with an antibody specific to SIX1 (rabbit polyclonal, M120, Santa Cruz) followed by HRP-conjugated goat anti-rabbit secondary antibody (SC-2004, Santa Cruz) and detected with ECL plus detection kit (GE Amersham). Protein loading was estimated with mouse anti-β-actin monoclonal antibody (AC-74, Sigma). Band intensities were quantitated with the Total Labs TL100 1D gel analysis software (Nonlinear).

Soft agar colony formation assay. One milliliter of 0.5% agarose in DMEM with 20% FBS was plated in each well of a twelve-well plate and left to set for 20 minutes. This layer was overlaid with 5000 HEK-293 cells stably expressing pSilencer-scramble or pSilencer-miR-185 in 2.5 mL of 0.34% agarose diluted in DMEM with 20% FBS. Cells were incubated as normal for 10 days and colonies were counted using the Total Labs TL100 colony counting software (Nonlinear).

Cell viability assay. HEK-293, MCF7 and SKOV3 cells were plated at 3000 cells per well in 96-well plates with triplicate wells for each transfection. Neutral red assay was performed according to standard protocols.

Flow cytometry. 20,000 HEK-293, MCF7 and SKOV3 cells stably overexpressing the pSilencer-scramble or pSilencer-miR 185 vector were fixed in cold ethanol, resuspended in Propidium Iodide solution (0.1% Triton-X/PBS, 2 mg DNAse-free RNAse A, 0.2 mg propidium iodide) and analyzed for cell cycle progression using Modfit software.

Apoptosis assay. Activation of caspase-3 and -7 was determined using the Caspase-Glo 3/7 assay kit (Promega) according to manufacturer's instructions. Briefly, 5000 MCF7 cells were plated in triplicate and transiently transfected with miR-185 mimic or miR-control as described above. Samples were read 1 hour after incubation with the caspase substrate 48 and 72 hours after serum starvation. For TRAIL-mediated apoptosis studies, 3000 SKOV3 cells were transfected with miRNA-control or miR-185 mimic for 48 hours followed by treatment with varying concentrations of full length recombinant TRAIL for 24 hours. Cell viability was measured as described above.

Tumorigenicity assays in nude mice. All experimental procedures involving animals were performed according to the institutional ethical guidelines for animal experiment. HEK-293 cells (2×10⁶) stably expressing pSilencer-miR-185 or -scramble were suspended in DMEM media and injected into the subrenal capsule of RAG2^{-/-}′ γc^{-/-} (purchased from Taconic Inc., Germantown, N.Y.) nude male mice (approx. 30 g bw) using a 50 μl glass syringe with a 22-gauge blunt needle (Hamilton Co., Reno, Nev.). Animals were sacrificed 24 days after transplantation and tumor volume was measured using the formula (L×D×W)×π/6, where the (L) is length, (D) is depth and (W) width.

### Example 1

This Example illustrates that miR-185 expression correlates reciprocally with Six1 gene expression in multiple human cancer cell lines and tumor tissues.

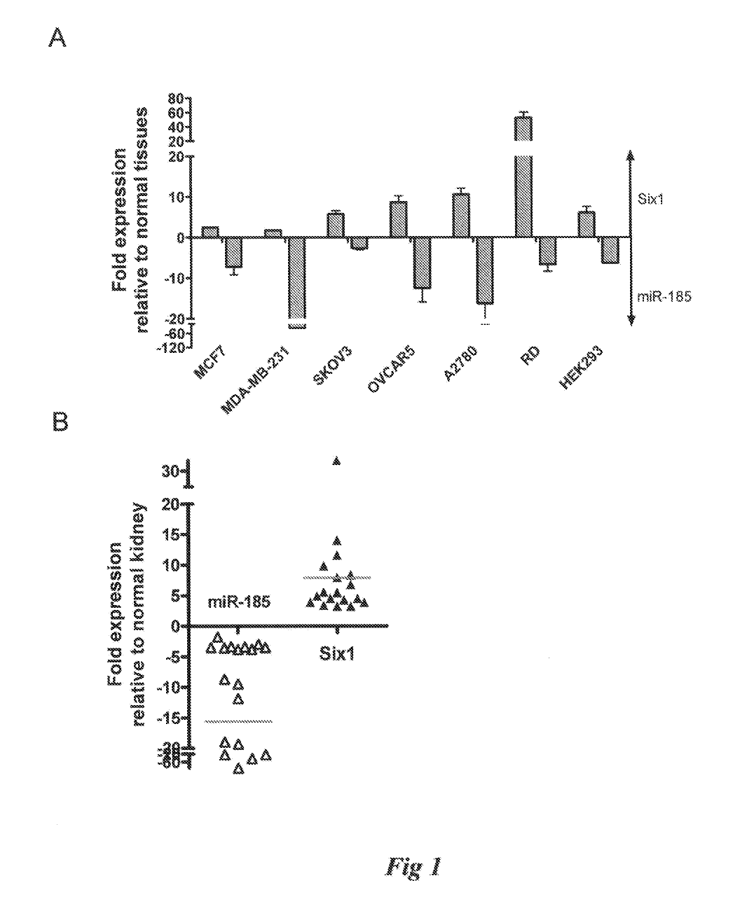

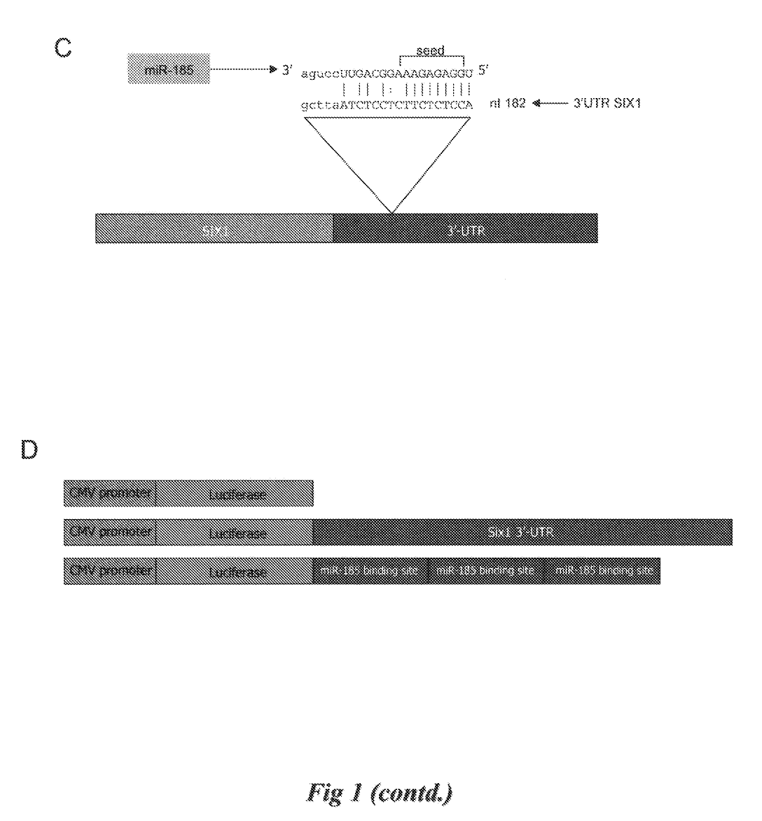

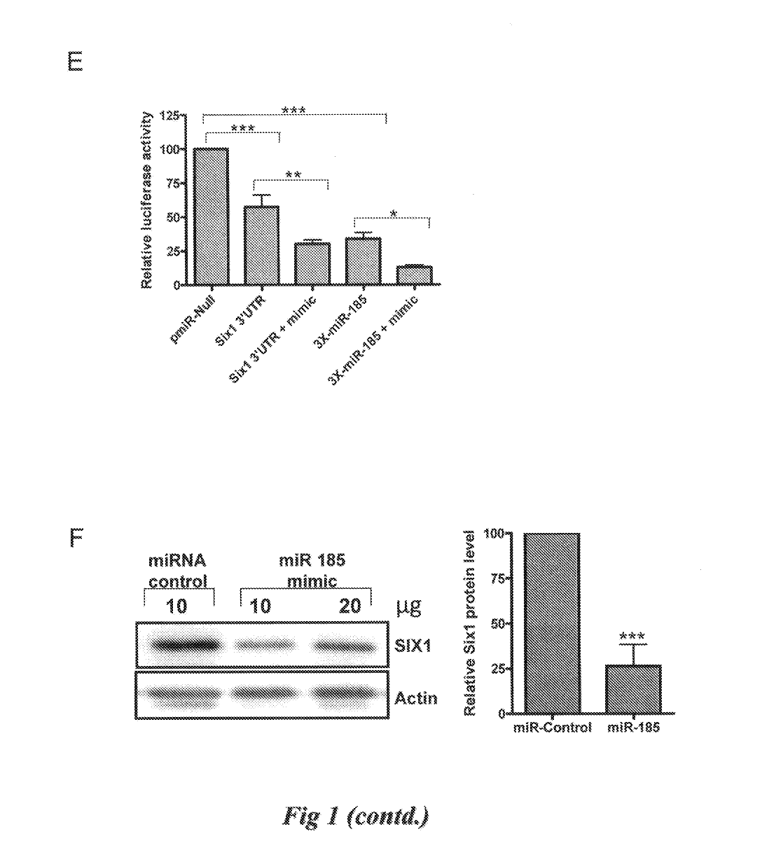

Target prediction algorithm TargetScan (http://www.targetscan.org/) predicted 16 miRNAs to target Six1. Out of these, miR-185 was predicted by six other prediction algorithms (including miRNAda, mirTarget2, miTarget, PITA, RNA22 and RNA hybrid) to regulate Six1. Furthermore, miR-185 was found to be evolutionary conserved throughout vertebrates, suggesting it to have an important function across a variety of species. Moreover, of all the predicted miRNAs, only miR-185 had perfect inverse correlation with Six1 expression in multiple human cancer cell lines including breast (MCF7 and MDA-MB-231), ovarian (SKOV3, OVCAR5 and A2780), rhabdomyosarcoma (RD) and kidney (HEK-293) when compared to their corresponding normal tissues (FIG. 1A). Real time PCR analysis shows inverse correlation between miR-185 and its putative target Six1 in multiple cancer cell lines: breast cancer (MCF7, MDA-MB-231), ovarian cancer (SKOV3, OVCAR5 and A2780) as well as rhabdomyosarcoma (RD) and human embryonic kidney (HEK-293) (FIG. 1A). The higher expression levels of Six1 and significantly lower expression of miR-185 observed in multiple cancer cell lines also corresponded with their expression in pediatric renal tumor tissues, suggesting misregulation of miR-185 to be a frequent event responsible for Six1 overexpression in human cancers (FIG. 1B). Wilms' and normal matched control kidney tissues (n=36, Children's Oncology Group (COG), Arcadia, Calif.) were studied. Total RNA was extracted from cell lines and their corresponding normal human adult tissues, and Wilms' tumors and normal matched control kidney tissues using the miRNEasy Mini kit (Qiagen). Two micrograms of total RNA was reverse transcribed using the miScript RT kit (Qiagen) and real-time PCR was performed with a forward primer specific to miR-185 or a primer set specific to Six1 using the miScript SYBR Green PCR kit (Qiagen). Replicate reactions were run for each cDNA sample. The relative expression of each gene was quantified by measuring ΔΔCt values and normalizing against RNU19 or 18S for miRNA or gene expression, respectively.

Collectively, the results described in this Example demonstrate that Six1 is a bonafide target of miR-185 and their reciprocal expression correlates strongly in human cancer cell lines and Wilms' tumors.

### Example 2

This Example illustrates that miR-185 targets the 3′UTR of the Six1 homeobox gene.

Because miRNAs generally regulate gene expression by binding to the 3′UTRs of their target genes, the present inventors predicted that miR-185 represses Six1 expression levels by binding to its 3′UTR. Bioinformatics analyses revealed that Six1 3′UTR contained one putative miR-185 binding site for miR-185 (FIG. 1C). FIG. 1C shows a Schematic of the putative miR-185 binding sequence in the Six1 3′UTR. To examine whether miR-185 indeed binds to Six1 3′UTR at its putative binding site, HEK-293 cells were transfected with pMIR-Report vector construct containing the Six1 3′ UTR (FIG. 1D) and luciferase activity was measured. For the pMIR-Six1 3′ UTR construct, the wild type 3′-UTR segment of the Six1 gene was amplified from genomic DNA and subcloned downstream of the Luciferase gene in the pmiR-Report vector (Ambion) at SacI and SpeI sites. To generate the miR-185 triplicate construct (3X-miR-185), an oligonucleotide containing the triplicated miR-185 binding sequence in the Six1 3′UTR (˜60 nt) was annealed and cloned in pMIR reporter vector. FIG. 1D shows a diagram of the luciferase reporter constructs (pmiR-null) containing either the 3′UTR of Six1, or triplicated miRNA binding sequences (3X-miR-185). As shown in FIG. 1E, luciferase activity was significantly repressed in pMIR-Six1 3′UTR transfected cells when compared to the null construct suggesting that Six1 expression levels may be regulated by miRNA/s binding to sequences within its 3′UTR. HEK-293 cells were plated at 55% confluency in six-well plates. PmiR-Report constructs were co-transfected with PRL-CMV using Lipofectamine 2000 as per manufacturer's instructions (Invitrogen). Luciferase activity was measured 48 hours after transfection using the dual luciferase reporter assay system (Promega). Firefly luciferase activity was normalized to Renilla luciferase activity for each transfected well. For overexpression studies, miRNA control or pre-miR-185 mimic were transfected sequentially after transfection of either pMIR-Six1 3′UTR or pMIR-3X-miR-185 luciferase constructs. Mean of four independent experiments (performed in duplicate for each experiment); bars, SEM. *, P<0.05; **, P<0.01; ***, P<0.001, comparison between two groups as indicated. Similar results were observed in breast (MCF7) and ovarian (SKOV3) cancer cell lines. FIG. 1E shows results providing validation of Six1 3′UTR as a miR-185 target. To further confirm our earlier findings, we determined the activity of a construct containing only the miR-185 binding sequence in triplicate (FIG. 1D). Results show that miR-185 triplicated binding sequence elicited a more robust translational repression of luciferase activity when compared to the full 3′UTR of Six1, which contains a single miR-185 binding site, confirming that this sequence is a bonafide miR-185 binding site (FIG. 1E). Further substantiating these findings, we found that luciferase activities of both the Six1 3′UTR- and triplicate miR-185 binding sequence-containing constructs were further repressed in cells overexpressing miR-185 mimic (FIG. 1e, right panel). Finally, we assessed the effect of miR-185 on Six1 protein levels by performing western blot analysis in the ovarian cancer cell line SKOV3, which is known to highly express Six1 (Behbakht et al., 2007). Overexpression of miR-185 mimic in SKOV3 resulted in significantly reduced Six1 protein expression levels (FIG. 1F). Western blot analyses of SKOV3 cells transfected with miR-185 mimic (left panel) using anti-SIX1 antibody (1:500; rabbit polyclonal, M120, Santa Cruz) followed by HRP-conjugated goat anti-rabbit secondary antibody (SC-2004, Santa Cruz) and detected with ECL plus detection kit (GE Amersham). Protein loading was estimated with mouse anti-β-actin monoclonal antibody (AC-74, Sigma). 10 and/or 20-t μg of each sample were resolved on a 12% (w/v) SDS-PAGE gel and transferred to a Millipore nylon membrane. Gel photograph is representative of four independent experiments. Band intensities were quantitated with the Total Labs TL100 1D gel analysis software (Nonlinear) (right panel); SEM. ****, P<0.001. Similar results were observed in MCF7 and HEK-293 cells. FIG. 1F shows results illustrating that miR-185 regulates SIX1 protein expression.

Collectively, the results described in this Example demonstrate that miR-185 regulates Six1 expression in human cancers by directly binding to its 3′UTR and eliciting translational repression.

### Example 3

This Example illustrates that miR-185 inhibits colony formation in vitro and tumor growth in vivo.

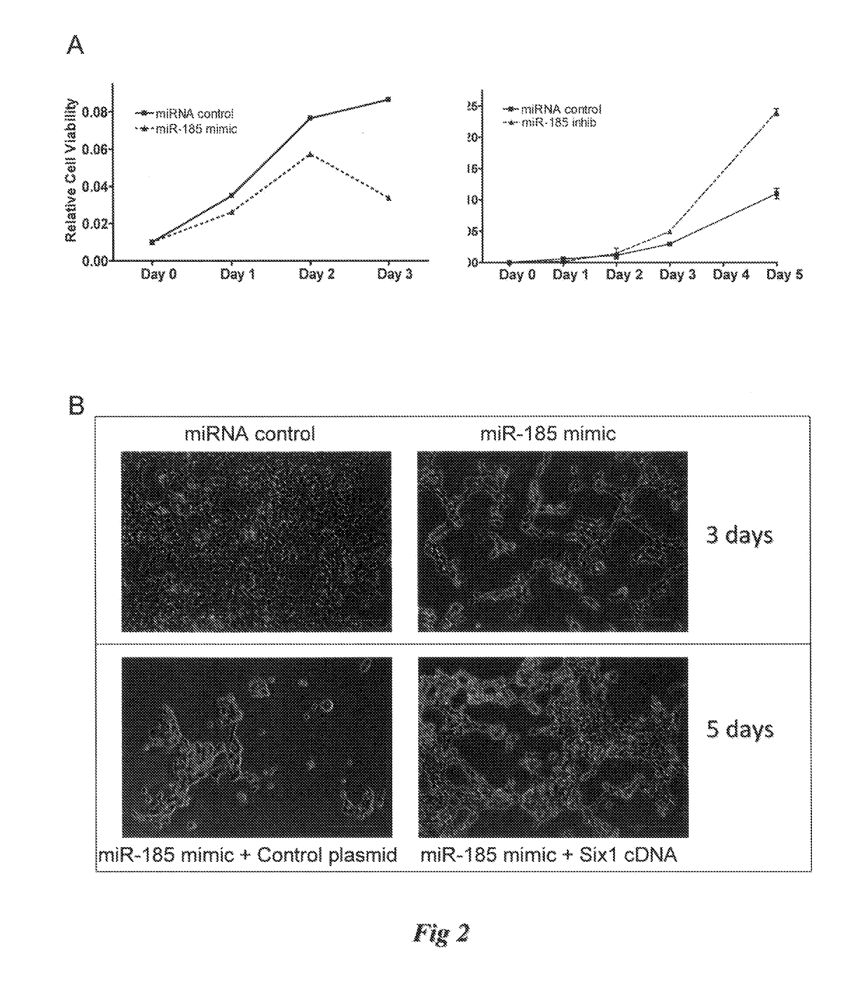

The significantly decreased expression of miR-185 in tumor tissues and multiple cell lines prompted us to examine the contribution of miR-185 in tumorigenesis. To determine this, we first examined the effect of miR-185 on cell proliferation, a property known to be promoted by Six1. miR-185 overexpression in HEK-293 cells resulted in significantly reduced cellular proliferation (FIG. 2A, left panel, and 2B, compare top panels), while inhibition of miR-185 led to increased cellular viability (FIG. 2A, right panel). Importantly, overexpression of Six1 cDNA restored the cell growth inhibition, suggesting that miR-185 specifically inhibits cell growth and viability through its repression of Six1 (FIG. 2B, compare bottom panels). FIG. 2A shows a cell proliferation assay of HEK-293 cells (3000 cells per well in 96-well plates) transfected with either miR-185 mimic (for 3 days) or miR-185 inhibitor (for 5 days) or miRNA control. Cell growth relative to miRNA control was measured by neutral red assay as described previously. Mean of at least three independent experiments (performed in triplicate for each experiment). Similar results were also observed in MCF7 and SKOV3 cells. FIG. 2A shows results illustrating that miR-185 regulates cell growth and survival through Six1. FIG. 2B shows a photomicrograph of HEK-293 cells (60,000 cells per well) transiently transfected either with miRNA control or miR-185 mimic for 3 days (compare top panels), and miR-185 overexpressing cells further transfected with a control or Six1 cDNA plasmid after 48 hours for 3 more days (compare lower panels). Photos were obtained using a Nikon LCD microscope (10× magnification) and are representative of 3 independent experiments. FIG. 2B illustrates that miR-185 overexpression inhibits cell proliferation.

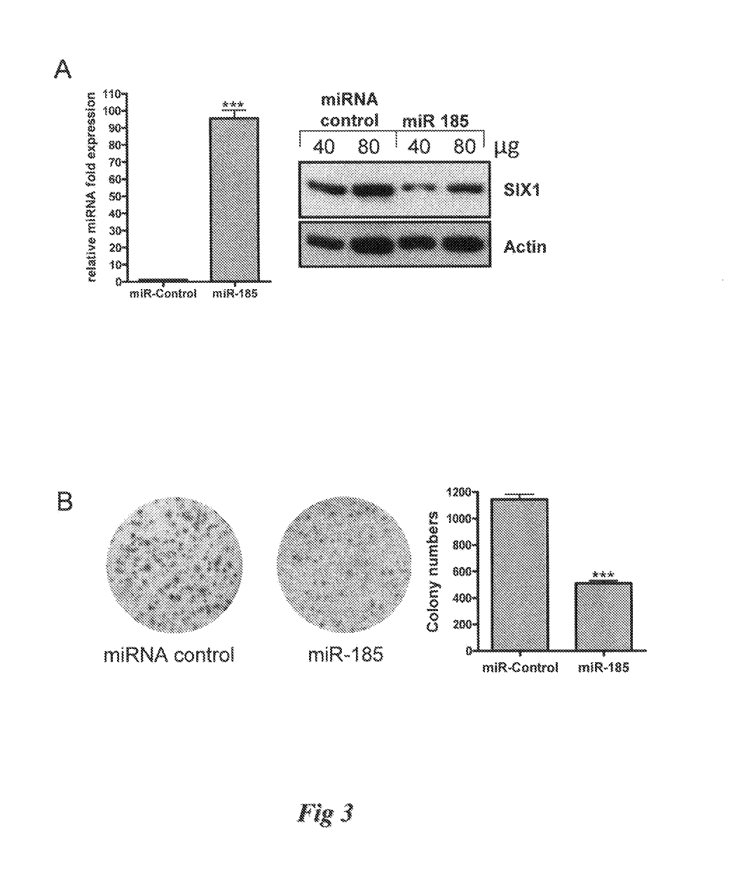

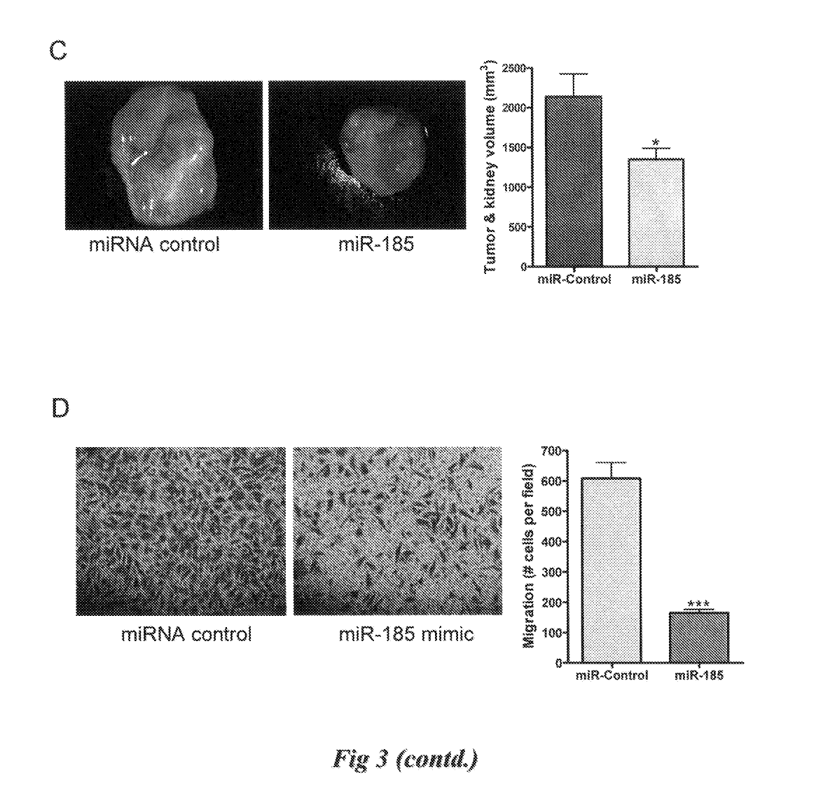

Next, soft-agar colony formation assays were performed to assess whether miR-185 inhibits anchorage independent growth. FIG. 3 illustrates that miR-185 inhibits anchorage independent growth in vitro and tumor growth in vivo. For in vitro and in vivo tumorigenicity assays the present inventors used miR-185-stably expressing HEK-293 cells passaged over 52 times, which are reported to be highly tumorigenic and cause tumor growth in immuno-compromised mice. miR-185 stably overexpressing cells showed significantly lower Six1 protein levels (FIG. 3A) and exhibited drastically reduced colony forming capacity (both in colony number and size) when compared to vector control, suggesting a potential tumor suppressor-like activity (FIG. 3B). FIG. 3A shows results obtained from real-time PCR using a miR-185 primer (left panel) and Western blot analysis using anti-Six1 antibody (1:500) (right panel) on HEK-293 cells stably expressing either pSilencer-scramble (miRNA Control) or pSilencer-miR-185 (miR-185). The pSilencer-miR-185 construct was generated by amplifying ˜450 bp pri-miR-185 genomic sequence from normal kidney genomic DNA (extracted with the Red Ex N Amp kit (Sigma)) and cloned in BamHI and HindIII sites of the pSilencer 4.1 puro vector. For the results shown in FIG. 3B, stable lines were then subjected to soft-agar colony formation assays. Briefly, 1 mL of 0.5% agarose in DMEM with 20% FBS was plated in each well of a twelve-well plate and left to set for 20 minutes. This layer was overlaid with 5000 stable HEK-293 cells in 2.5 mL of 0.34% agarose diluted in DMEM with 20% FBS. Cells were incubated as normal for 10 days and colonies were counted using the Total Labs TL100 colony counting software (Nonlinear). The results shown in FIG. 3B are average of three independent experiments, bars, SEM. ****, P<0.001. To confirm these findings in vivo, control and miR-185 stable HEK-293 cells were injected into the kidney capsule of nude mice and tumor growth was evaluated 24 days after injection. In sharp contrast to the tumors from control transfectants, miR-185 over-expressing tumors were dramatically reduced in size (FIG. 3C). FIG. 3C illustrates that overexpression of miR-185 inhibits tumor formation in nude mice. HEK-293 cells (2×10⁶) stably over-expressing either pSilencer-scramble (miRNA Control) or pSilencer-miR-185 (miR-185) were injected into the subrenal capsule of RAG2^{-/-}′ γc^{-/-} (purchased from Taconic Inc., Germantown, N.Y.) nude male mice (approx. 30 g bw) using a 50 μl glass syringe with a 22-gauge blunt needle (Hamilton Co., Reno, Nev.). Animals were sacrificed 24 days after transplantation and tumor volume was measured using the formula (L×D×W)×π/6, where the (L) is length, (D) is depth and (W) width. Photographs show representative features of tumor growth (left panel). Bar graph showing mean tumor+kidney volume for mice injected with miR-185 transfectants (n=4) and scramble-transfectants (n=4) (right panel), bars, SEM. **, P<0.05. It is worth noting that compared to vector control the actual tumor size (without kidney) in miR-185 overexpressing xenograft will likely be much smaller, as kidneys in the vector control xenograft were found to be compressed and significantly smaller due to extensive tumor growth, while miR-185 overexpressing xenograft kidneys were less affected and comparatively larger (FIG. 3C, data not shown). Since Six1 is reported to be a critical metastatic regulator, we also determined the effect of miR-185 on cell migration, as the ability of tumor cells to migrate is a prerequisite for tumor metastasis. miR-185 overexpression drastically reduced the migration capability of SKOV3 cells in a Transwell chemotaxis assay (FIG. 3D). FIG. 3D shows a transwell cell migration assay of SKOV3 cells transfected with a control miRNA or miR-185 mimic. 48 hours after transfection, cells were trypsinized, resuspended in serum free media and loaded into the top of a 3 μm pore Transwell chamber. Serum-containing media was placed in the bottom chamber as a chemoattractant and cells were incubated at 37° C. and allowed to migrate through the chemotaxis chamber for 24 hours. The migrated cells on the bottom of the chamber were fixed with 10% Formalin and stained with 0.4% Crystal Violet for 3 hours. Similar tumor suppression and suppression of tumor cell metastasis results (not shown) were achieved when from about 10 to about 200 nM range miR-185 was introduced into tumor cells. Experiments were repeated in triplicate wells and migrated cells were counted microscopically (200×) in five different fields per filter. SEM. ****, P<0.001.

Collectively, the results described in this Example demonstrate that miR-185 has properties consistent with that of a potent tumor suppressor.

### Example 4

This Example illustrates that miR-185 alters cell-cycle progression and sensitizes cells to apoptosis.

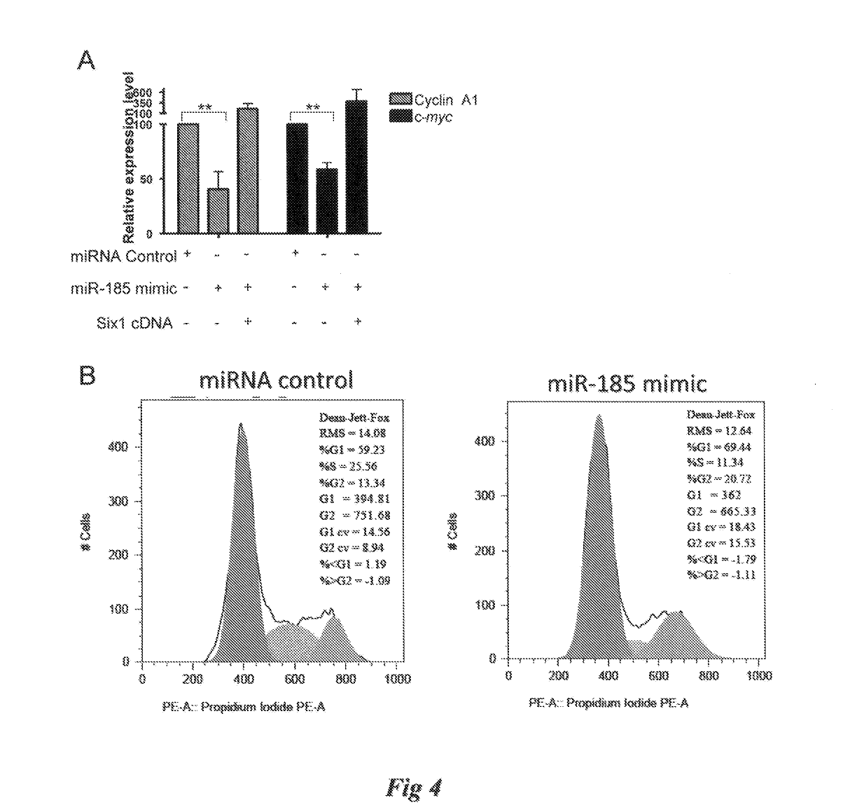

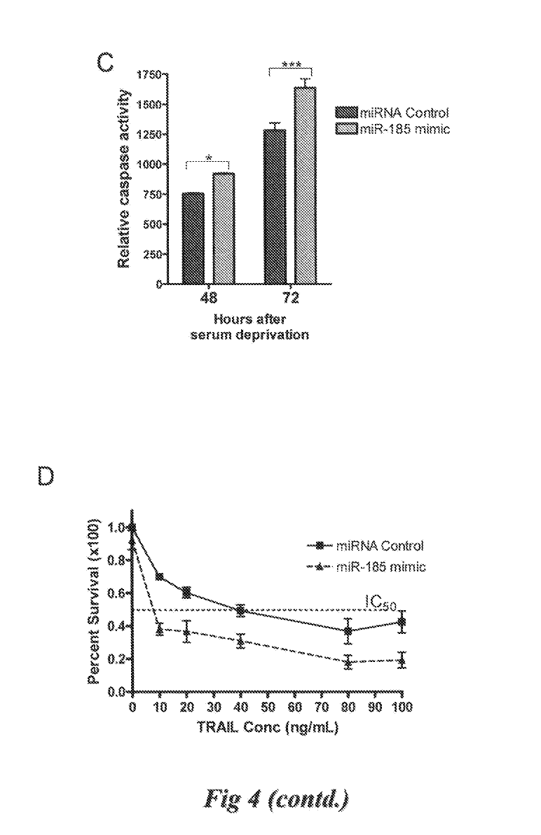

To directly address Six1's involvement in miR-185-regulated anti-proliferative and tumor-suppressor activity, we examined the effect of mir-185 on the expression levels of the Six1 transcriptional targets and cell-cycle regulators cyclin A1 and c-myc (Coletta et al., 2004; Yu et al., 2006). FIG. 4 illustrates that miR-185 alters cell-cycle progression and sensitizes cells to apoptosis through Six1. We found that miR-185 over-expression resulted in significantly decreased levels of cyclin A1 and c-myc, which were rescued when Six1 was overexpressed, in all tumor cell lines tested (FIG. 4A, data not shown). FIG. 4A illustrates that Six1 overexpression rescues miR-185 inhibited expression of cyclin A1 and c-myc. Quantitative real-time PCR analysis on total RNA isolated from SKOV3 cells transfected first with miRNA control or miR-185 mimic for 48 hrs and subsequently with either Six1 cDNA expression construct (+Six1) or pBluescript KS⁺ control (−Six1) for an additional 24 hrs, using primers specific for cyclin A1 and c-myc. Since Six1 overexpression has been shown to attenuate the DNA damage-induced G2 check point, we next asked whether mir-185 affects cell cycle progression. FIG. 4B shows PI staining of HEK-293 cells transiently transfected with pSilencer-miR-185 (miR-185) shows an increase in number of cells in G0/G1 and a decrease in S phase as compared to cells expressing pSilencer-scramble (miRNA Control). Briefly, 20,000 transfected HEK-293 cells were fixed in cold ethanol, resuspended in Propidium Iodide solution (0.1% Triton-X/PBS, 2 mg DNAse-free RNAse A, 0.2 mg propidium iodide) and analyzed for cell-cycle progression using Modfit software. Similar results were also obtained in SKOV3 cells. (c and d) miR-185 overexpression increases cellular sensitivity to apoptosis. PI-staining of miR-185 over-expressing HEK-293 revealed an increase in G1 cell populations and a decrease in cells in S phase (FIG. 4B) suggesting a block in G1/S phase of cell cycle. These findings indicate that miR-185 may mediate its anti-proliferative and tumor suppressor-like activity in part by altering the cell cycle progression and inhibiting expression of cell cycle regulators and Six1 transcriptional targets cyclin A1, and c-myc, inappropriate activation of which are known to promote cell proliferation and tumor growth.

To further understand the mechanism of miR-185 tumor suppressor activity, the present inventors investigated the effect of miR-185 on apoptosis, as evasion of apoptosis is a crucial event during malignant transformation. Activation of caspase-3 and -7 was determined using the Caspase-Glo 3/7 assay kit (Promega) according to manufacturer's instructions. Briefly, 5000 MCF7 cells were plated in triplicate and transiently transfected with miR-185 mimic or miR-control as described above. Samples were read 1 hour after incubation with caspase substrate 48 and 72 hours after serum starvation. FIG. 4C illustrates that miR-185 increases the sensitivity of serum-starved cells to apoptosis. Interestingly, we found that compared to miRNA control-transfected cells, miR-185 overexpression resulted in significantly increased caspase-3/7 activity in serum starved cancer cells (FIG. 4C). Since adaptation to low nutrition is presumed to be one of the prerequisites for cancers cells to survive in their tumor microenvironment, our results showing that miR-185 sensitizes cancers cells to serum starvation-induced apoptosis suggests a critical role for miR-185 in this process. Furthermore, to specifically address the role of Six1 in miR-185-mediated apoptosis, the present inventors determined the effect of miR-185 on the TRAIL apoptotic pathway, as constitutively higher Six1 expression is known to confer resistance to TRAIL-mediated apoptosis. FIG. 4D illustrates the effect of TRAIL on the survival of SKOV3 cells transfected with miRNA control (solid line) or mir-185 mimic (dotted line). 3000 SKOV3 cells were transfected with miRNA-control or miR-185 mimic for 48 hours followed by treatment with varying concentrations of full length recombinant TRAIL for 24 hours. Cell viability was assessed by neutral red assay. Shown are the mean of at least three independent experiments, bars, SEM. **, P<0.05; ***, P<0.001, comparison between two groups as indicated. Cell survival studies revealed that SKOV3 cells transfected with miR-185 were drastically more sensitive to TRAIL (IC₅₀˜7 ng/ml) compared with SKOV3 cells transfected with miRNA control (IC₅₀˜40 ng/ml) (FIG. 4D). This is a significant finding as Six1 overexpressing TRAIL-resistant cancers have poor outcome and TRAIL has been proposed to affect several aspects of tumorigenesis including inhibition of tumor growth and metastasis, surveillance against tumor growth and response to chemotherapy.

Collectively the results described in this example illustrate that strategies to use miR-185 as a therapeutic regimen are expected not only to result in slower tumor growth and decreased tumor invasiveness but to lead to increased sensitivity of cancer cells to TRAIL-induced apoptosis.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

It is therefore, contemplated that the claims will cover any such modifications or embodiments that fall within the true scope of the invention.

What is claimed is:

1. A method of modulating gene expression in a cell, comprising administering to the cell isolated and/or chemically synthesized miR-185 in an amount sufficient to modulate the expression of a tumor-causing gene.

2. The method according to claim 1 wherein the tumor-causing gene comprises Six1.

3. The method according to claim 1, wherein the cell is a cancer cell.

4. The method according to claim 3, wherein the cell is in a tumor susceptible to the tumor-causing gene.

5. The method according to claim 1, wherein the amount is effective to suppress tumor growth.

6. The method according to claim 1, wherein the amount is effective to suppress tumor metastasis.

7. The method according to claim 1, wherein tumor is drug resistant and the amount is effective to sensitize the tumor to cell death.

8. The method according to claim 7, wherein the amount is effective to sensitize the tumor to a tumor necrosis factor-related apoptosis inducing ligand.

9. The method according to claim 1, wherein the miR-185 is in a pharmaceutical formulation.

10. The method according to claim 1, wherein the method further comprising administering the miR-185 in conjunction with a tumor necrosis factor-related apoptosis inducing ligand.

11. A method of treating a patient in need thereof, comprising administering to the patient in need thereof, a pharmaceutical formulation comprising isolated and/or chemically synthesized miR-185 in a therapeutically effective amount sufficient to modulate cellular expression of a tumor-causing gene.

12. The method according to claim 11, wherein the tumor-causing gene comprises Six1.

13. The method according to claim 11, wherein the amount is effective to suppress tumor growth.

14. The method according to claim 11, wherein the amount is effective to suppress tumor metastasis.

15. The method according to claim 11, wherein the patient has a drug resistant tumor and the amount is effective to sensitize the tumor to cell death.

16. The method according to claim 15, wherein the amount is effective to sensitize the tumor to a tumor necrosis factor-related apoptosis inducing ligand.

17. The method according to claim 11, wherein the miR-185 is in a pharmaceutical formulation.

18. The method according to claim 10, wherein the method further comprising administering the miR-185 in conjunction with a tumor necrosis factor-related apoptosis inducing ligand.

19. A tumor suppressor, comprising isolated and/or chemically synthesized miR-185 in an amount sufficient to modulate expression of a tumor-causing gene.

20. A method of treating a patient in need thereof, comprising administering to the patient in need thereof, a pharmaceutical formulation comprising isolated and/or chemically synthesized miR-185 in a therapeutically effective amount sufficient to modulate cellular expression of a tumor-causing gene.


SEQUENCE LISTING (2 sequences)

SEQ ID NO: 1 - RNA, 22 nt, Homo sapiens
uggagagaaaggcaguuccuga

SEQ ID NO: 2 - RNA, 82 nt, Homo sapiens
agggggcgagggauuggagagaaaggcaguuccugaugguccccuccccaggggcuggcuuuccucugguccuucccuccca